(19) United States
(12) Patent Application Publication
(10) Pub. No.: US 2006/0190059 A1
(43) Pub. Date: Aug. 24, 2006

(54) Cochlear Stimulation Device

(76) Inventor: Glen A. Griffith, Newbury Park, CA (US)

Correspondence Address: HENRICKS SLAVIN AND HOLMES LLP;SUITE 200, 840 APOLLO STREET, EL SEGUNDO, CA, 90245, US

(21) Appl. No.: 11/379,779

(22) Filed: Apr. 21, 2006

Related U.S. Application Data
(63) Continuation-in-part of application No. 11/124,495, filed on May 5, 2005.
(60) Provisional application No. 60/568,957, filed on May 7, 2004.

(51) Int. Cl. A61N 1/18

(52) U.S. Cl. 607/57

(57) ABSTRACT

A cochlear stimulation apparatus includes an implantable device and an external device. The implantable device includes a receiving coil, an array of electrodes configured to be fitted within the cochlea of a user, and circuitry for receiving signals through the receiving coil and generating stimulation pulses that are directed to selected electrodes of the array. The external device is in the form of a single, integral unit. The external device includes circuitry for processing sensed sound information to generate the signals, a transfer coil for transferring the signals to the receiving coil and a ferromagnetic core for providing a low reluctance path between the transfer coil and the receiving coil.

### CROSS-REFERENCE TO RELATED APPLICATIONS

This application is a continuation-in-part of U.S. patent application Ser. No. 11/124,495, filed on May 5, 2005, which claims the benefit of U.S. Provisional Application No. 60/568,957, filed on May 7, 2004, which application is incorporated herein by reference.

### FIELD OF THE INVENTION

The present invention relates to cochlear stimulation systems, and more particularly to a cochlear stimulation system that does not require a headpiece or an implanted magnet and that improves inductive coupling between external and internal coil components of the cochlear stimulation system.

### BACKGROUND OF THE INVENTION

Current cochlear implant systems include an implant portion and an external portion. The implant portion typically includes: (1) an electrode array, (2) an implanted coil, and (3) a hermetically-sealed housing to which the electrode array and implanted coil are attached and in which electronic circuitry, e.g., data processing circuitry and pulse generator circuitry, is housed. The external portion typically includes: (1) a microphone, (2) a power source (e.g., a battery), (3) electronic circuitry for processing the signals sensed by the microphone and for generating control and other signals that are transmitted to the implant portion, and (4) a headpiece, connected to the electronic circuitry by way of a cable or wire(s), in which an external coil is housed. In operation, the headpiece coil (external coil) is inductively coupled with the implanted coil so that power and data can be transferred to the implant portion from the external portion.

Some cochlear implant systems have the implanted coil carried within the hermetically-sealed housing; while other cochlear implant systems have the implanted coil carried outside of the hermetically sealed housing. In either type of system, it is necessary that the external coil be carefully aligned with the implanted coil so that maximum coupling efficiency can be achieved between the external coil and the implanted coil, thereby allowing power and data to be transferred transcutaneously through the headpiece coil to the implanted coil with which it is aligned.

The alignment between the headpiece coil and the implanted coil is achieved through the use of a magnet or other type of mechanical device. Typically, a magnet is carried within the implant portion and physically centered within the implanted coil. Another magnet, or material that is attracted to the implanted magnet, is carried within the headpiece and centered within the headpiece coil so that the headpiece is attracted to the implanted magnet, and held in place over the implanted magnet by magnetic attractive forces. As the headpiece is so held, the two coils—the implanted coil and the headpiece (or external) coil—are maintained in a substantially optimally aligned position.

Disadvantageously, the headpiece, although small, is sometimes viewed as cumbersome and unsightly. Further, because the headpiece coil is usually held in place magnetically, the magnetic forces can sometimes prove uncomfortable, i.e., too strong, or cause physical irritation requiring intervention, so spacers or other means must be utilized to find a magnetic force that is sufficiently strong to hold the headpiece in place, yet not so strong as to be uncomfortable. Additionally, the presence of the magnet within the implant portion of the system may prevent or potentially interfere with desired or needed medical procedures, e.g., Magnetic Resonance Imaging (MRI).

Further, the headpiece, with its accompanying cable that connects the headpiece to the external circuitry, and the magnet, or other material that is attracted to the implanted magnet, and the implanted magnet used in the implant portion of the system, all represent separate parts of the cochlear implant system which contribute in a significant way to the overall cost and reliability of the system.

It would be helpful to be able to provide a cochlear implant system that does not require an external coil housed in a headpiece, with its attendant extra parts and reduced reliability, and which is held in place over an implanted coil by a magnetic force created through the use of an implanted magnet, which implanted magnet also represents an additional part and creates through its use its own set of potential undesirable attributes. It would also be helpful to be able to provide a cochlear implant system with a single external unit or component.

It would also be helpful to be able to provide a cochlear stimulation system that does not require a headpiece or an implanted magnet with improved inductive coupling between external and internal coil components of the cochlear stimulation system.

### BRIEF SUMMARY OF THE INVENTION

The present invention addresses the above and other needs by integrating the transfer coil (i.e., the external coil) in the body or housing of the external portion of the cochlear implant system. For example, when the speech processor is carried within a behind-the-ear (BTE) module that is worn by a user of the cochlear implant system, the transfer coil is carried within the BTE module or housing, or formed as part of the ear hook used to hold the BTE module in place.

Thus, the present invention—with the external transfer coil forming an integral part of the external portion of the system—does not require a separate headpiece. This means that the present invention also does not require the use of an implanted magnet. Hence, the present invention may be described as a headpieceless and magnetless cochlear implant system. In an example embodiment, a cochlear implant system includes an external device (e.g., a single external unit or component) provided with a transfer coil (e.g., integrally formed therein), and an implanted device with a receiving coil, or other means for communicating with the external device.

In an example embodiment, a cochlear implant system includes an implanted portion and an external portion. In this example embodiment, the external portion that includes a microphone for sensing sound, an external housing for enclosing electrical circuitry and a power source, sound processing circuitry within the external housing for processing signals generated by the microphone in response to sound sensed through the microphone or otherwise applied to the sound processing circuitry as an input signal, signal processing circuitry within the external housing for processing the input signal and generating stimulation, control and power signals for transferring to the implanted portion, and an external coil, affixed to the external housing, for coupling the stimulation, control and power signals to the implanted portion.

In an example embodiment, the implanted portion includes an implanted coil inductively coupled with the external coil, electronic circuitry for receiving through the implanted coil the stimulation, control and power signals, an electrode array having a multiplicity of electrode contacts adapted to be placed within the cochlea of a user, and a pulse generator for generating stimulation pulses that are directed to selected electrode contacts within the electrode array as controlled by the control signals.

In an example embodiment, the external coil is integrally formed as part of the external housing. In another example embodiment, the external coil is carried within the external housing.

In an example embodiment, the external housing includes a behind-the-ear (BTE) unit. In another example embodiment, the external coil is carried within the BTE unit.

In an example embodiment, the external housing includes an earhook. In another example embodiment, the external coil is integrally formed as part of the earhook.

In an example embodiment, the external housing includes a behind-the-ear (BTE) unit with an earhook for holding the BTE unit in place behind the ear of a user. In another example embodiment, the cochlear implant system further includes a stem attached to the earhook, and the microphone is attached to the stem and adapted to be positioned within the concha area surrounded by the pinna of a user's ear.

In another example embodiment, the external housing includes a behind-the-ear (BTE) unit that is held in place behind the ear of a user with an earhook that is integrally attached to the external housing. In such embodiment, the external coil may be integrally formed as part of the external housing and/or as part of the earhook. Also, in such embodiment, the microphone may be included within, or attached to, the external housing, or attached to a stem that is connected or attached to the external housing. Such stem, when used, places the microphone within the concha area surrounded by the pinna of the user's ear, thereby positioning the microphone near the ear cannel where sound is naturally collected.

In another example embodiment, the transfer coil is placed into an in-the-canal speech processor. In such embodiment, the external housing is, for example, a small cylindrical-shaped housing that is adapted to be positioned in the ear canal.

In another example embodiment, the implanted coil is implanted such that the implanted coil and the external transfer coil overlap axially and remain in relatively close proximity. In such embodiment, the implanted coil is sufficiently large to accommodate surgical technique, anatomical variation, tissue growth, and maintain a sufficient coupling coefficient for the required efficiency and reliability.

In an example embodiment, a cochlear stimulation apparatus includes an implantable device and an external device. The implantable device includes a receiving coil, an array of electrodes configured to be fitted within the cochlea of a user, and circuitry for receiving signals through the receiving coil and generating stimulation pulses that are directed to selected electrodes of the array. The external device is in the form of a single, integral unit, and includes circuitry for processing sensed sound information to generate the signals and a transfer coil for transferring the signals to the receiving coil.

In an example embodiment, the receiving coil and the transfer coil overlap axially, and the receiving coil is sufficiently large to be inductively coupled with the transfer coil.

In an example embodiment, the external device includes a behind-the-ear (BTE) unit. In an example embodiment, the transfer coil is contained within the BTE unit.

In an example embodiment, the external device includes an ear hook. In an example embodiment, the transfer coil is integrally formed as part of the ear hook.

In an example embodiment, the external device includes a cylindrical-shaped housing adapted to be positioned in the ear canal of the user.

In an example embodiment, a cochlear stimulation apparatus includes an implantable device including electrodes configured to be fitted within the cochlea of a user and circuitry for processing signals to generate stimulation pulses that are directed to the electrodes, an external device including circuitry for processing sensed sound information to generate the signals, and means for communicating the signals from the external device to the implantable device.

In an example embodiment, the external device includes a behind-the-ear (BTE) unit and/or an ear hook.

In an example embodiment, the external device includes a cylindrical-shaped housing adapted to be positioned in the ear canal of the user.

In an example embodiment, the external device is a single, integral unit.

In an example embodiment, the means for communicating includes a transfer coil that is electrically connected to the circuitry for processing sensed sound information and inductively coupled to the implantable device.

In an example embodiment, a cochlear stimulation apparatus includes an implantable device including a receiving coil, an array of electrodes configured to be fitted within the cochlea of a user, and circuitry for receiving signals through the receiving coil and generating stimulation pulses that are directed to selected electrodes of the array, and an external device in the form of a single, integral unit, the external device including circuitry for processing sensed sound information to generate the signals, the external device including a transfer coil for transferring the signals to the receiving coil and a ferromagnetic core for providing a low reluctance path between the transfer coil and the receiving coil.

In an example embodiment, the external device includes a housing that is symmetrical in shape such that the external device can be used for either the left or right ear. In an example embodiment, the ferromagnetic core includes ends that are positioned at substantially equal distances from opposite inside walls of the housing.

In an example embodiment, the transfer coil surrounds the ferromagnetic core at a center portion of the ferromagnetic core.

In an example embodiment, the ferromagnetic core has a length of approximately 1 cm or less.

In an example embodiment, the receiving coil includes two symmetrical lobes.

In an example embodiment, the receiving coil is larger than the transfer coil. In an example embodiment, the size disparity between the coils is sufficiently large to provide a flattened coupling coefficient response in response to an increase in distance between the coils.

In an example embodiment, the external device includes a behind-the-ear (BTE) unit. In an example embodiment, the transfer coil is contained within the BTE unit.

In an example embodiment, a cochlear stimulation apparatus includes an implanted portion, and an external portion that includes a microphone for sensing sound, a behind-the-ear (BTE) unit for enclosing electrical circuitry and a power source, the BTE unit being symmetrical in shape such that the BTE unit can be used for either the left or right ear, sound processing circuitry within the BTE unit for processing signals generated by the microphone in response to sound sensed through the microphone or otherwise applied to the sound processing circuitry as an input signal, signal processing circuitry within the BTE unit for processing the input signal and generating stimulation, control and power signals for transferring to the implanted portion, and an external coil within the BTE unit for coupling the stimulation, control and power signals to the implanted portion.

In an example embodiment, the BTE unit includes an earhook.

In an example embodiment, the external portion includes a ferromagnetic core for providing a low reluctance path between the external coil and the implanted portion. In an example embodiment the ferromagnetic core includes ends that are positioned at substantially equal distances from opposite inside walls of the BTE unit. In an example embodiment, the external coil surrounds the ferromagnetic core at a center portion of the ferromagnetic core. In an example embodiment, the ferromagnetic core has a length of approximately 1 cm or less.

In an example embodiment, the implanted portion includes an implanted coil inductively coupled with the external coil, electronic circuitry for receiving through the implanted coil the stimulation, control and power signals, an electrode array having a multiplicity of electrode contacts adapted to be placed within the cochlea of a user, and a pulse generator for generating stimulation pulses that are directed to selected electrode contacts within the electrode array as controlled by the control signals. In an example embodiment, the implanted coil includes two symmetrical lobes. In an example embodiment, the implanted coil is larger than the external coil. In an example embodiment, the size disparity between the coils is sufficiently large to provide a flattened coupling coefficient response in response to an increase in distance between the coils.

In an example embodiment, a cochlear stimulation apparatus includes an implantable device including electrodes configured to be fitted within the cochlea of a user, circuitry for processing signals to generate stimulation pulses that are directed to the electrodes, and a receiving coil, and an external device including circuitry for processing sensed sound information to generate the signals, the external device including a transfer coil for communicating the signals from the external device to the implantable device, the transfer coil being inductively coupled to the receiving coil, wherein the receiving coil is sufficiently larger than the transfer coil to provide a flattened coupling coefficient response in response to an increase in distance between the coils.

In an example embodiment, the external device includes a ferromagnetic core for providing a low reluctance path between the transfer coil and the receiving coil. In an example embodiment, the transfer coil surrounds the ferromagnetic core at a center portion of the ferromagnetic core. In an example embodiment, the ferromagnetic core has a length of approximately 1 cm or less. In an example embodiment, the ferromagnetic core includes ends that are positioned at substantially equal distances from opposite inside walls of the external device.

In an example embodiment, the receiving coil includes two symmetrical lobes.

In an example embodiment the external device includes a housing that is symmetrical in shape such that the external device can be used for either the left or right ear.

In an example embodiment, the external device includes a behind-the-ear (BTE) unit.

In an example embodiment, the external device includes an ear hook.

In an example embodiment, the external device is a single, integral unit.

Various advantages are potentially achieved through use of the present invention. These advantages include, but are not limited to, reduced cost, improved cosmetics, improved reliability, elimination of the headpiece, a smaller-sized implant unit which requires no magnet, a reduced incision size during surgery when implanting the implanted portion, a carrier signal having a frequency legally allowed by regulatory agencies, and improved performance. In various embodiments, a fully implantable one-piece system may last up to 20 years or more.

### BRIEF DESCRIPTION OF THE DRAWINGS

The above and other aspects of the present invention will be more apparent from the following more particular description thereof, presented in conjunction with the following drawings wherein:

FIG. 1 is a block diagram of an implantable stimulation system, such as the implantable cochlear stimulation system of the present invention;

FIG. 2 depicts an electrode array that is used with an implantable cochlear stimulation system;

FIG. 3 shows a behind-the-ear (BTE) external speech processor coupled to a headpiece, as is used with cochlear stimulation systems of the prior art;

FIG. 4 illustrates a headpieceless BTE external speech processor positioned behind the ear of a user in accordance with an example embodiment of the present invention;

FIG. 5 schematically illustrates various components of a Micro System, which is an example form of a headpieceless and magnetless cochlear implant system, in accordance with an example embodiment of the present invention;

FIG. 6 schematically provides an overview of the Micro BTE of FIG. 5 and a Micro Implantable Cochlear Stimulator (ICS) configured in accordance with an example embodiment of the present invention;

FIG. 7 is a block diagram of the Micro ICS of FIG. 6, showing the various input and output signals applied thereto, or received therefrom;

FIG. 8 is a functional block diagram of the Micro ICS of FIGS. 6 and 7;

FIG. 9 shows a functional block diagram of the Micro BTE of FIGS. 5 and 6;

FIG. 10 illustrates various example Micro BTE configuration options in accordance with example embodiments of the present invention;

FIG. 11 depicts a block diagram of the connectivity module of FIG. 10;

FIG. 12 schematically shows the components of a Micro Fully Implantable Stimulation (FIS) system;

FIG. 13 is a block diagram of a Micro FIS system made in accordance with an example embodiment of the present invention;

FIG. 14A is a side view of a cochlear stimulation apparatus that includes an external device with a transfer coil and a ferromagnetic core in accordance with an example embodiment of the present invention;

FIG. 14B is a back view of the cochlear stimulation apparatus of FIG. 14A;

FIG. 15A illustrates field lines for the transfer coil of FIG. 14 without the ferromagnetic core;

FIG. 15B illustrates field lines for the transfer coil of FIG. 14 with the ferromagnetic core;

FIG. 16 is a perspective view of an implantable device with a D-shaped receiving coil in accordance with an example embodiment of the present invention;

FIG. 17A is a perspective view of the D-shaped receiving coil of FIG. 16;

FIG. 17B is a bottom view of the D-shaped receiving coil of FIG. 16; and

FIG. 17C is a side view of the D-shaped receiving coil of FIG. 16.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

The following U.S. patents and U.S. Publication teach various features and elements and systems that may be used with a cochlear implant system embodying the present invention. Each of the listed U.S. patents or U.S. Publication is incorporated herein by reference: U.S. Pat. Nos. 5,584,869; 6,181,969; 6,212,431, 6,219,580; 6,272,382; 6,308,101; 6,505,076; and Pub. No. 2003/0031336 A1.

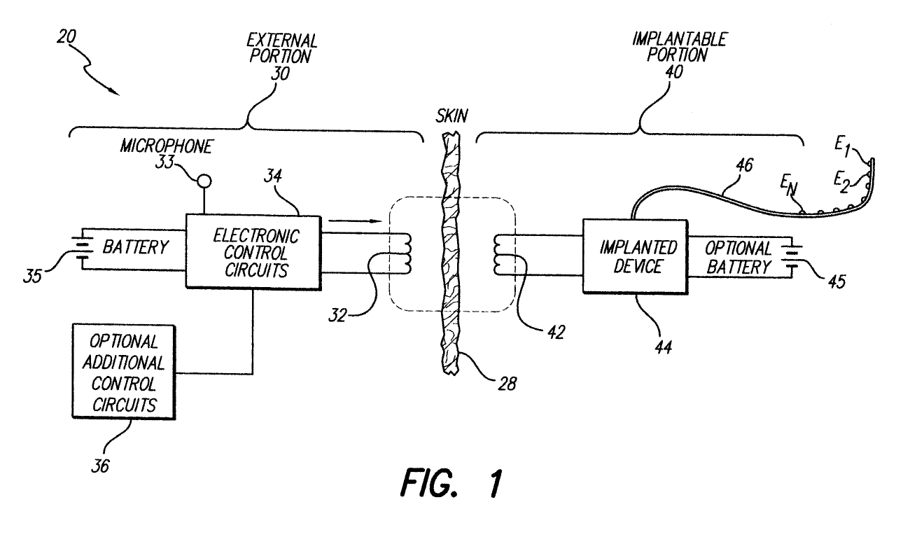

FIG. 1 shows an implantable stimulation system 20, e.g., an implantable cochlear stimulation system, according to an example embodiment of the present invention. The system 20 includes an external portion 30 and an implantable portion 40. In this example embodiment, the implantable portion 40 includes an implanted coil 42 for receiving data, control and power signals from an external transmitter. The implanted coil 42 is connected to an implanted device 44, e.g., an Implantable Cochlear Stimulator (ICS), which implanted device 44 houses appropriate signal processing and pulse generation circuitry. An optional battery 45 may be included as part of, or coupled to, the implanted device 44. Also connected to the implanted device 44 is an electrode array 46 having a multiplicity n of spaced-apart electrode contacts, E1, E2, . . . En, located at or near its distal end. The number of electrode contacts n varies depending upon the circumstances, but typically n is at least 8, and may be 16 or higher, e.g., 32, for a cochlear implant device.

Still referring to FIG. 1, in this example embodiment, the external portion 30 includes electronic control circuits 34 (e.g., inside a case or housing). A microphone 33 provides a source of input signals for the electronic control circuits 34. A battery 35 provides operating power for the circuitry contained within the external portion 30 of the cochlear implant system, and also for the electronic circuits contained within the implanted device 44. In an embodiment where the implantable portion 40 utilizes a battery 45 which is rechargeable to help provide its operating power, the external battery 35 can provide the power needed to recharge the rechargeable battery 45.

Optional additional control circuits 36 can also be used for providing optional input/control signals to the electronic control circuits 34 of the external portion 30. An example of optional input signal is an audio signal from an external source, such as a radio, CD, cell phone, MP3 player, or TV. Also by way of example, an optional control signal can be a programming signal to help configure the operation of the circuits included within the electronic control circuits 34 or the electronic circuits included within the implanted device 44.

When implanted, the implantable portion 40 of the cochlear implant system 20 is separated from the external portion 30 by a layer of skin 28. Thus, the data, control and power signals are transmitted from the external coil (or transfer coil) 32 and coupled transcutaneously through the layer of skin 28 (and other tissue) to the implanted coil (or receiving coil) 42.

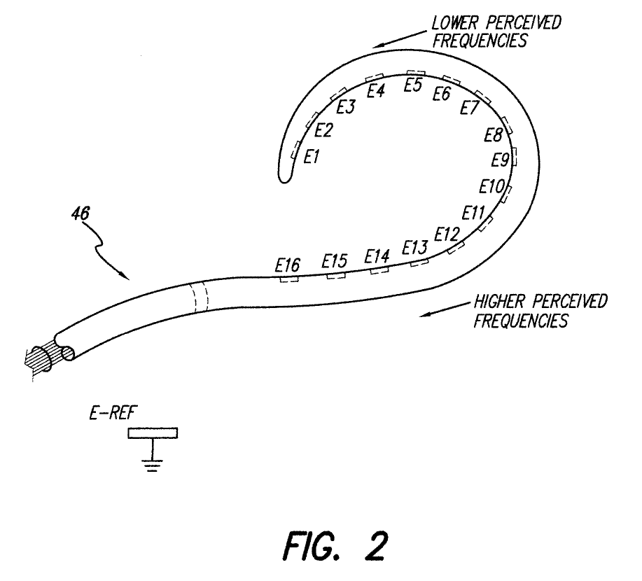

FIG. 2 depicts the distal end of one type of an electrode array 46 that can be used with the implantable stimulation system 20 (e.g., an implantable cochlear stimulation system). As seen in FIG. 2, in this example embodiment, the array 46 includes an in-line configuration of sixteen electrodes contacts, designated E1, E2, E3, . . . E16 E16. Electrode contact E1 is the most distal electrode contact, and electrode contact E16 is the most proximal. The more distal electrode contacts, e.g., the electrode contacts having lower numbers such as E1, E2, E3, E4, are the electrode contacts through which stimulation pulses are applied in order to elicit the sensation of lower perceived frequencies. The more proximal electrode contacts, e.g., the electrode contacts having higher numbers such as E13, E14, E15 and E16, are the electrode contacts through which stimulation pulses are applied in order to elicit the sensation of higher perceived frequencies. The particular electrode contact, or combination of electrode contacts, through which stimulation pulses are applied is determined by the speech processing circuitry, which circuitry, inter alia, and in accordance with a selected speech processing strategy, separates the incoming sound signals into frequency bands and analyzes how much energy is contained within each band, thereby enabling it to determine which electrode contacts should receive stimulation pulses.

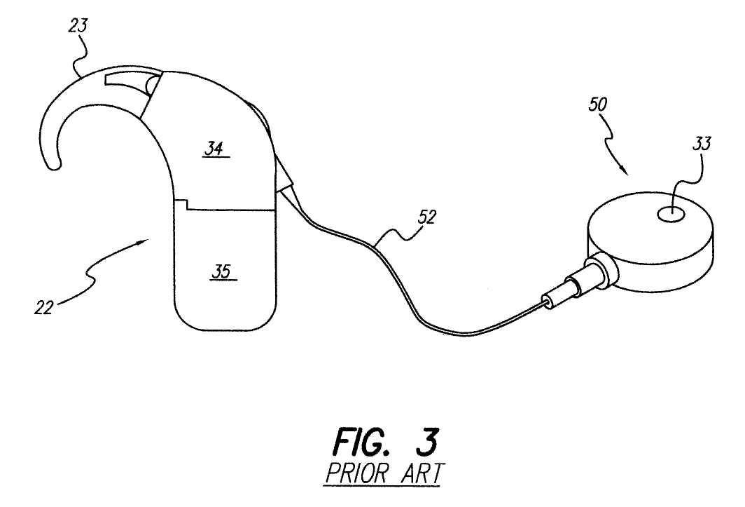

FIG. 3 shows a conventional behind-the-ear (BTE) external speech processor coupled to a headpiece 50 via a cable 52, as is used in cochlear stimulation systems of the prior art. In such prior systems, the microphone 33 is typically housed within the headpiece 50. The external coil 32 (not shown in this figure) is also housed within the headpiece 50. A BTE unit 22 includes the electronic control circuits 34, e.g., sound processing circuits, as well as a battery 35. Additionally, an ear hook 23 provides a means (or mechanism) for holding the BTE unit 22 behind the ear of a user.

Advantageously, in various embodiments of the present invention, the headpiece 50 is eliminated. Without the headpiece 50, and coupling cable 52, the system includes fewer parts, and is thus rendered more reliable, more efficient, and portrays a better overall cosmetic appearance.

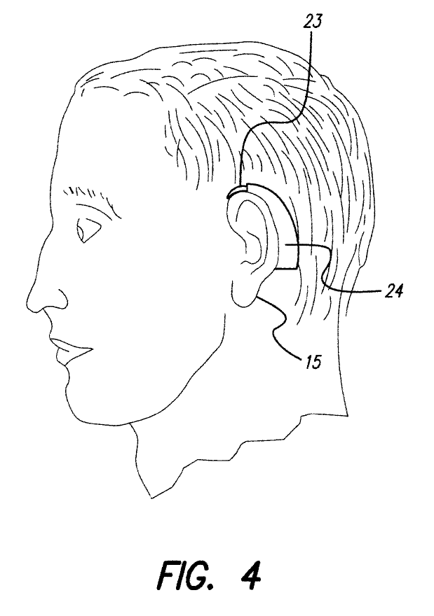

FIG. 4 illustrates a headpieceless BTE external sound processor 24 positioned behind the ear 15 of a user in accordance with an example embodiment of the present invention.

Because the headpieceless and magnetless system of the present invention allows the system to be much smaller than prior art systems, a headpieceless BTE external sound processor 24 in accordance with various embodiments of the present invention may also be referred to as a “Micro BTE” (where “Micro” refers to its relatively small size). Similarly, a magnetless Implantable Cochlear Stimulator 44 in accordance with various embodiments of the present invention may be referred to as a “Micro ICS”.

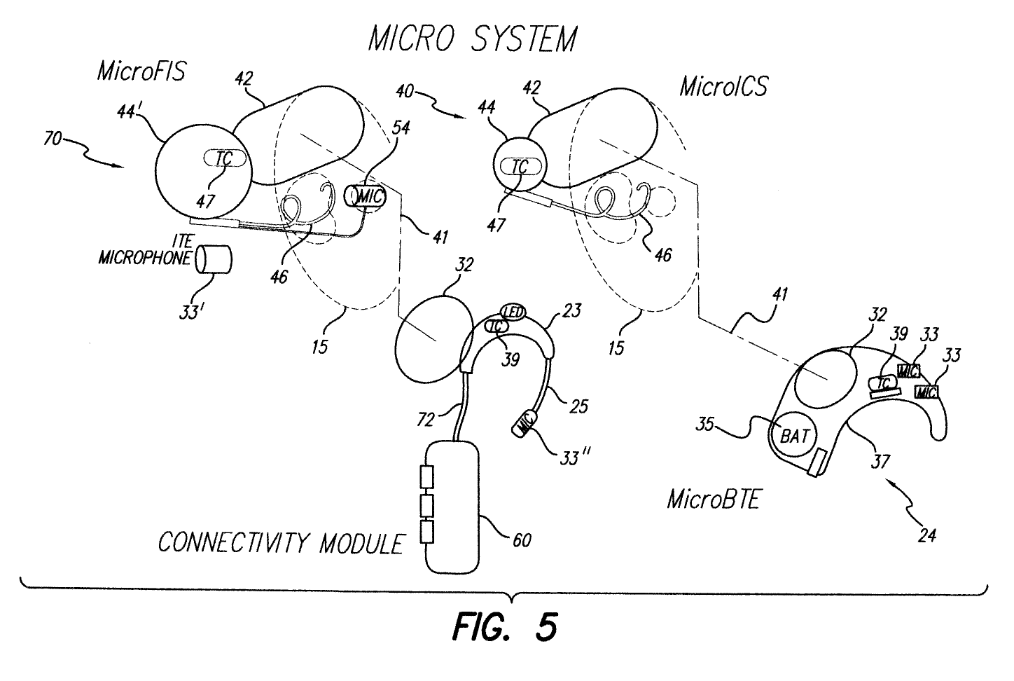

FIG. 5 schematically illustrates various components of a Micro System, an example embodiment of a headpieceless and magnetless cochlear implant system according to the present invention. In this example embodiment, the MicroICS 40 includes an ICS 44, an electrode array 46, a telecoil (TC) 47, and a receiving coil 42. (Dotted lines symbolically represent portions of the user's ear 15, or concha, or ear cannel, or cochlea.) In this example embodiment, the MicroBTE 24 includes a battery 35, one or more microphones 33, a telecoil (TC) device 39, and a transfer coil 32. In this example embodiment, the transfer coil 32 is embedded, or otherwise attached to, or made an integral part of, the BTE housing 37. Accessories can be mounted to the BTE housing 37, as desired, e.g., along a bottom edge thereof.

In another example embodiment, TC 47 is omitted and a reflected impedance monitoring technique (such as described in U.S. Pat. No. 6,212,431) is used as a means for communicating with the external device. For example, a resistor is electrically connected to the receiving coil 42 and a switch used to short the resistor to ground, and changes in the reflected impedance are sensed at the transfer coil 32. Other techniques can also be used to modulate a carrier signal that is inductively coupled between the transfer coil 32 and the receiving coil 42.

If two microphones 33 are used within the MicroBTE 24, then such microphones can advantageously be used to provide a directional microphone array. By way of example, the battery 35 includes a Lithium Ion battery or a Zinc Air battery.

Still with reference to FIG. 5, it is seen that when the MicroICS 40 is implanted, the axis of the receiving coil 42 is more or less (e.g., substantially) aligned with the axis of the external coil 32. Such axes are represented in FIG. 5 by the dotted-dashed line 41. In this example embodiment, the receiving coil 42 is relatively large in size compared to the ICS 44. However, the incision made to implant the ICS 44 need not be very big, because the coil 42 may be flexible, and can be squeezed through a small incision, and then spread out once through the incision. In various example embodiments, an implant unit (e.g., “can”) is also sufficiently small in size to be inserted through a small incision.

As further seen in FIG. 5, an example embodiment of a fully implantable stimulation (FIS) system is schematically depicted as a MicroFIS system 70. In this example embodiment, the system 70 includes a MicroICS 44′ (e.g., provided with a rechargeable battery that will last 15-20 years). A receiving coil 42 is attached to the MicroICS 44′, as is an electrode array 46. The MicroICS 44′ includes a telecoil 47 (e.g., a built-in telecoil) or equivalent means for communicating with an external device. Also used with the MicroICS 44′ is an implanted microphone 54, e.g., a middle ear microphone. In addition, an in-the-ear (ITE) microphone 33′ can be employed with the MicroFIS system 70. By way of example, the ITE microphone 33′ is a RF-coupled microphone that is placed in the ear canal, and is sometimes referred to as an in-the-canal (ITC) microphone.

In this example, an external coil 32 coupled or attached to an ear hook 23 is used with the MicroFIS system 70. In this example embodiment, the ear hook 23 is detachably connected, via cable 72, with a connectivity module 60. One of the main purposes of the connectivity module 60 is to allow recharging of the battery included within the MicroICS 44′. That is, if the battery within the MicroICS 44′ is charged, the MicroFIS system 70 shown in FIG. 5 can function without any external components. However, in various embodiments, the external components, including the external coil 32, and connectivity module 60, are used to, inter alia, recharge the battery. Such external components can also be used to provide auxiliary microphones, such as a T-Mic 33″ connected to the end of a stem 25 attached to the ear hook 23, as shown in this example embodiment. An example of the T-Mic 33″ is described in the previously cited U.S. Patent Publication. The advantage of using a T-Mic 33″ at the end of stem 25 is that it can be positioned near the center of the concha of the ear, which is the location where sound waves are naturally collected and funneled by the shape on the pinna of the ear. The sound signals sensed through the T-Mic 33″ can be transferred to the MicroFIS system 70 through a separate channel established between the external telecoil (TC) 39 and the implanted TC 47. Alternatively, if the connectivity module 60 is attached to the ear hook 60, the sound signals sensed through the T-Mic 33″ can be transferred to the MicroFIS system 70 through modulation of a carrier signal that is inductively coupled between the external coil 32 and the implanted coil 42.

As described previously in connection with the operation of the MicroICS system 40 and the MicroBTE 24, during operation, the external coil 32 and the implanted coil 42 of the MicroFIS system 70 have their respective axes aligned, as represented symbolically by the dotted-dashed line 41.

In an example embodiment, the connectivity module 60 can advantageously function as a body worn micro speech processor, which speech processor may be compatible with, e.g., the HiRes90K or the CII Bionic Ear, speech processors made by Advanced Bionics Corporation of Valencia, Calif. In an example embodiment, the connectivity module 60 also functions, as described previously, as a charger for the MicroFIS system 70. In an example embodiment, the connectivity module 60 additionally includes a backup microphone. The connectivity module 60 can also include a fitting interface, for example, via a Bluetooth or USB interface. In an example embodiment, the connectivity module 60 can also function as a telecoil remote control.

Advantageously, no magnets are used with the MicroFIS system 70 or the MicroICS system 40. Thus, such systems are magnetless and, as such, MRI compatible.

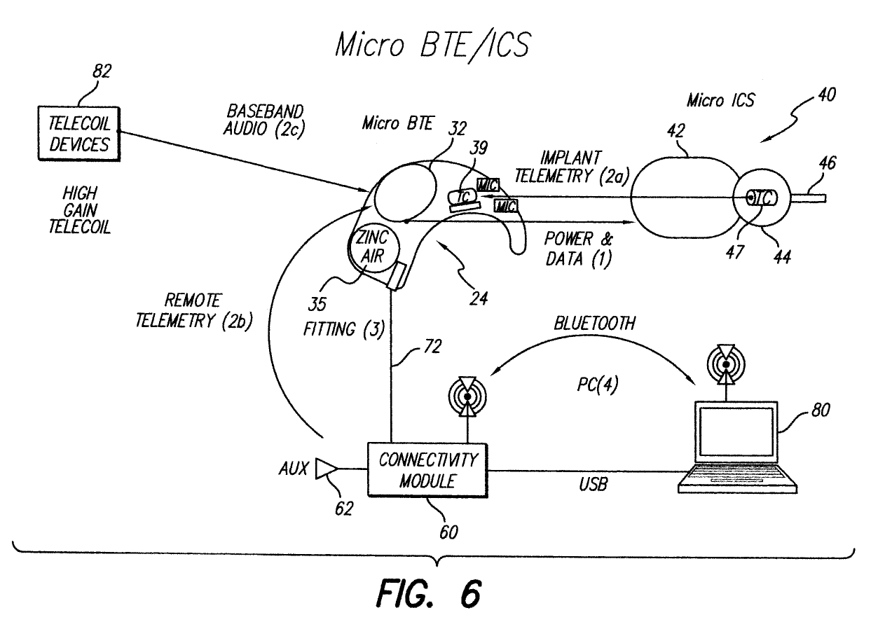

FIG. 6 shows additional details relative to the MicroBTE 24 and the MicroICS system 40. In this example overview, various communication links that can be established between components of the system are illustrated.

Power and data can be transmitted from the external coil 32 to the implanted coil 42, by way of example, at 27 MHz with 16-ary 500 Kbit Frequency Shift Keying (FSK) modulation, or Minimum Shift Keying (MSK) or other modulation scheme. The range for such transmission is only about one centimeter (cm), which means the external coil 32 must reside on or near the outer surface of the skin 28 (FIG. 1), and the implanted coil 42 must reside within about 1 cm of the inside surface of the skin.

In this example embodiment, various telecoil (TC) communication channels are shown. A first TC channel (2a) provides for implant telemetry and allows communications from the implanted TC 47 to the external TC 39. For example, first TC channel (2a) is an analog FM channel, with modulation ranging from about 200 Hz to 10 KHz. The range is about 1 cm. A second TC channel (2b) provides for remote telemetry and allows communication from the connectivity module 60 to the MicroBTE 24. For example, second TC channel (2b) is also an analog FM channel, with modulation at about 300 bps. The range is about 25 cm. A third TC channel (2c) provides a baseband audio channel from an external telecoil device 82 to the MicroBTE 24, for example, at frequencies ranging from about 200 Hz to 20 KHz.

In an example embodiment, the connectivity Module 60 connects to the MicroBTE 24 via interface 72, e.g., a 3-wire cable, which in this example is denoted Fitting (3). One wire is used for Power/Data-In/Clock. A second wire is used for Aux-In/Data-Out. A third wire is used for Ground. In the example embodiment shown in FIG. 6, the connectivity module 60 has an Auxiliary Input Port 62. This port can be used to input audio signals from numerous devices, such as a cell phone, a TV, a radio, a CD player, or the like.

In the example embodiment shown in FIG. 6, a personal computer (PC) 80 communicates with the connectivity module 60, e.g., via a standard USB or wireless Bluetooth connection, denoted PC (4). Such PC links facilitate the use of fitting and diagnostic programs defined by software loaded on the PC.

In this example embodiment, the MicroSystem operates without implant status through telemetry allowing the telecoil channel to be used for external telecoil devices and telecoil remote during normal operation. In an example embodiment, the telecoil is used for fitting and objective measures and external telecoil systems are shut down during the fitting process.

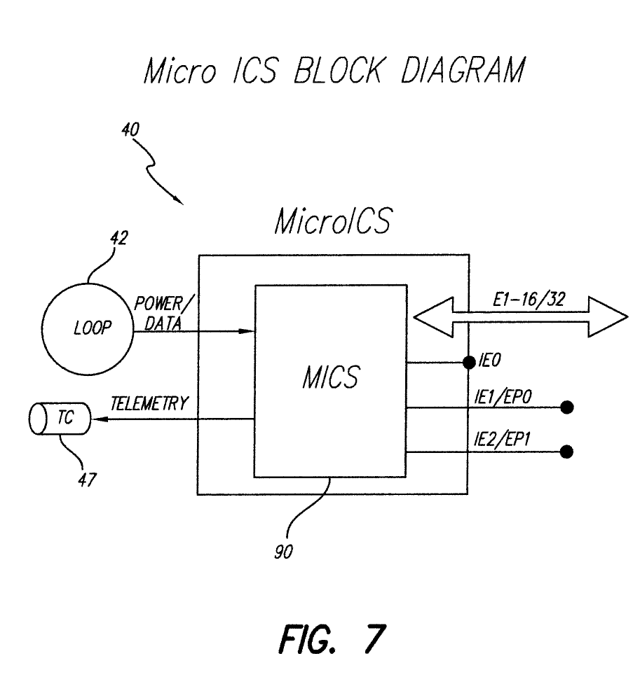

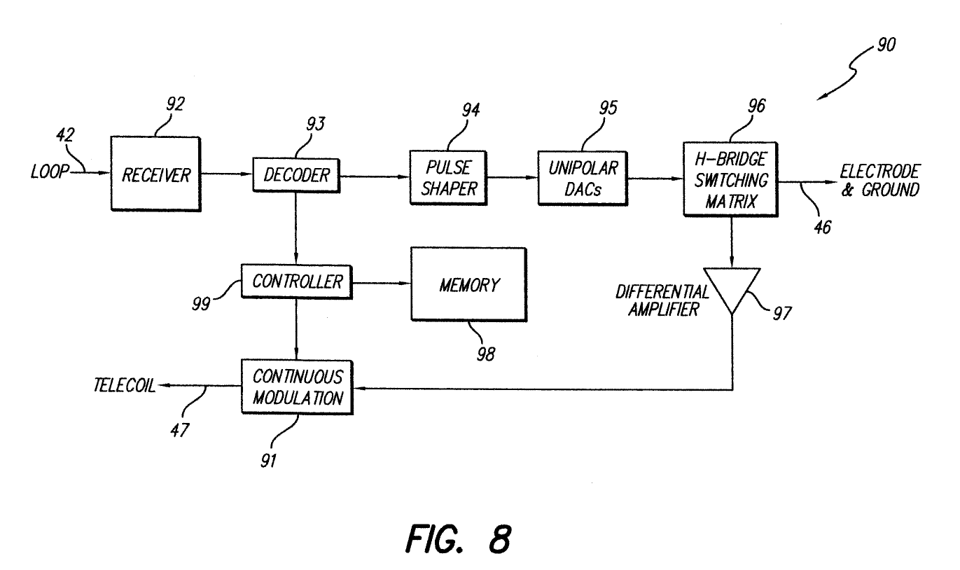

FIG. 7 is a block diagram of the Micro ICS system 40, showing the various input and output signals applied thereto, or received therefrom. In an example embodiment, the MicroICS is implemented using a MICS chip(s) 90 containing circuitry for performing the functions shown in the functional block diagram of FIG. 8.

FIG. 8 is a functional block diagram of an example embodiment of the Micro ICS system 40. In this example embodiment, the MICS chip(s) include circuitry that performs the following functions. A receiver 92 (e.g., 16-ary FSK or MSK) is connected to the implanted coil loop 42. The receiver output is directed to a decoder circuit 93. The decoder 93 sends a decoded signal to pulse shaper circuitry 94, after which it is sent to unipolar DACs (digital-to-analog converters) 95. The DACs 95 are connected to the electrode array 46 through an H-bridge switching matrix 96, which switching matrix allows bi-directional current to be sent to any selected electrode contact.

In this example embodiment, a secondary output of the decoder 93 is directed to a controller 99, which is controlled by one of three programs stored in a memory 98. The controller 99 controls the operation of the MICS 90 based on the programs stored in the memory 98. The controller 99 also controls a continuous modulation circuit 91, which modulates a signal representative of the pulses applied to the electrode contacts, sensed through a differential amplifier 97, which is applied to the implanted telecoil 47. Such signal transmitted through the telecoil 47 allows various parameters, such as impedance, associated with the operation of the MICS 90, to be monitored.

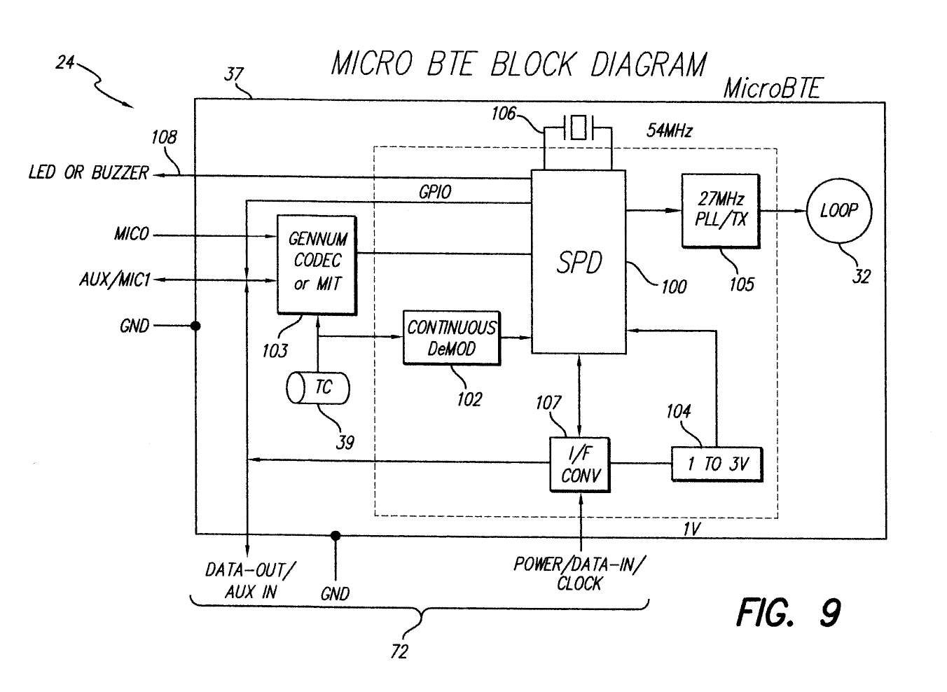

FIG. 9 shows a functional block diagram of an example embodiment of the MicroBTE 24. In this example embodiment, the MicroBTE 24 includes a low voltage (e.g., 1 volt) Signal Processor, Digital (SPD) chip 100. By way of example, the SPD 100 uses a 54 MHz clock signal, generated using a crystal 106, and a 27 MHz phase lock loop (PLL) transmitter circuit 105 drives the external coil 32. Such external coil 32, in an example embodiment, is integral with the housing 37 of the MicroBTE 24. Microphone or other input signals are processed in analog front end circuitry 103. In this example embodiment, telecoil 39 applies any signals that it senses to the analog front end circuitry 103 and also to continuous demodulation circuitry 102. The output of the continuous demodulation circuitry 102 is monitored (e.g., continuously) for commands and interrupts. If the SPD 100 determines that such commands and interrupts are valid, then it responds as required. Power/Data-In/Clock signals received from the connectivity module 60 (FIG. 6) over cable 72, are applied to IF Converter circuitry 107. One output of the converter circuitry 107 is directed to the SPD 100. Another output is applied to a voltage converter circuit 104 (e.g., 1-to-3 volt). A LED or Buzzer signal 108 is generated by the MicroBTE 24 to provide visual and/or audible status indicators to the user regarding the operating status of the MicroBTE.

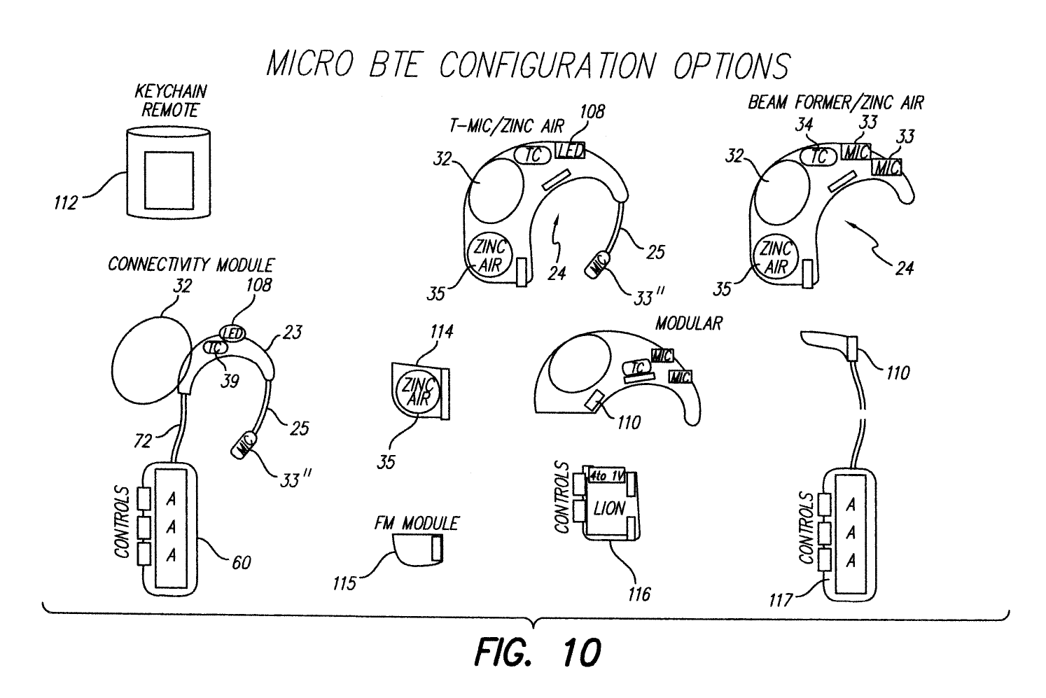

FIG. 10 illustrates examples of configuration options that can be used with the MicroBTE 24. Such options include: a keychain remote control 112; a T-Mic 33″ attached at the end of a stem 25; the use of two microphones 33 that allow a directionality of sound (beam former) to be used; a connectivity module 60 attached to an ear hook 23, wherein the connectivity module includes, e.g., a standard AAA battery; and various modules that attach to a bottom side of the MicroBTE 24, or to a connector 110 located along a bottom side of the MicroBTE 24. Such attachable modules include, e.g., a zinc air battery module 114, a FM module 115, a Lithium Ion battery module 116, and a connectivity module 117.

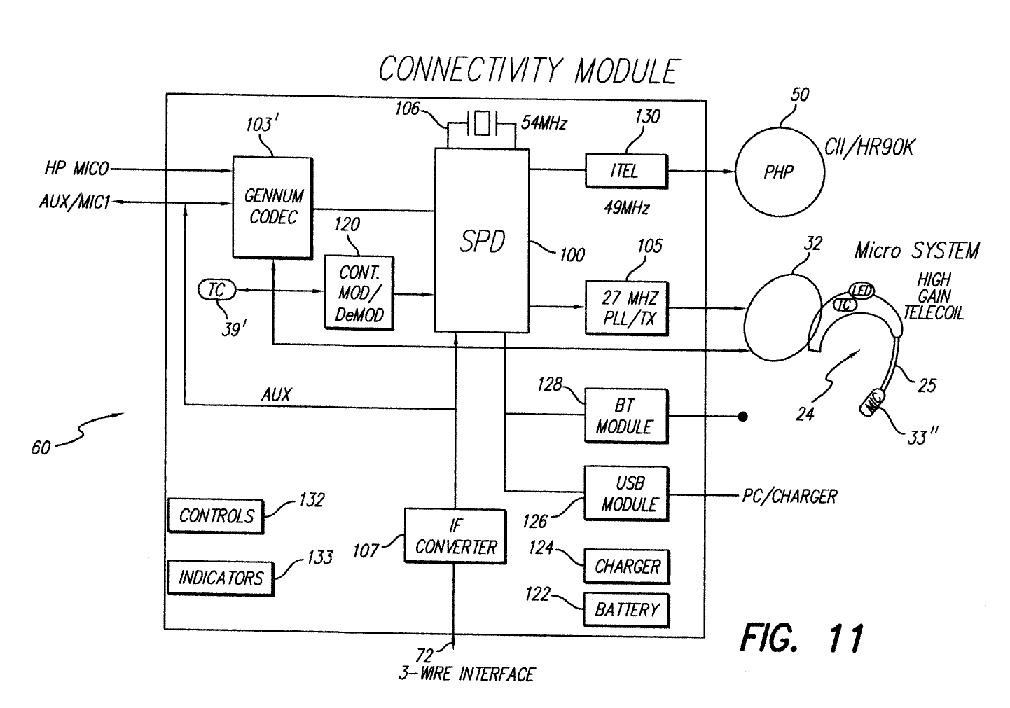

FIG. 11 depicts a block diagram of an example embodiment of the connectivity module 60. In this example embodiment, much of the circuitry contained within the connectivity module 60 can be the same as that used in the MicroBTE 24, in which case the same reference numerals are used to designate such common circuitry. The connectivity module 60 can be used to connect with the MicroBTE 24, as described above, or to connect with a headpiece 50 used with an existing cochlear implant system, such as a CII Bionic Ear system or a HiRes 90K system, made by Advanced Bionics Corporation.

In this example embodiment of the connectivity module 60, the SPD 100 uses a 54 MHz crystal clock 106, and IF converter circuitry 107 provides a three-wire interface 72 that can connect with the MicroBTE 24. An USB module 126, or a BlueTooth (BT) Module 128, allows communications with a remote PC. An internal and replaceable battery 122 provides operating power for the connectivity module 60. A charger circuit 124 allows power to be sent to the rechargeable battery included within the MicroFIS system 70 (FIGS. 5, 11 and 12). Analog front end circuitry 103′ interfaces with an auxiliary microphone or other external signal source. A telecoil 39′ provides for communications with external devices, such as a remote control, or with the MicroBTE 24. Signals received or sent through such Telecoil 39′ are modulated or demodulated by continuous modulation/demodulation circuitry 120. ITEL circuitry 130 facilitates a proper interface with the headpiece 50, when a connection with an existing cochlear implant system is required. Various controls 132 and indicators 133 allow the connectivity module to be adjusted, as needed, and to monitor its status and performance, as desired.

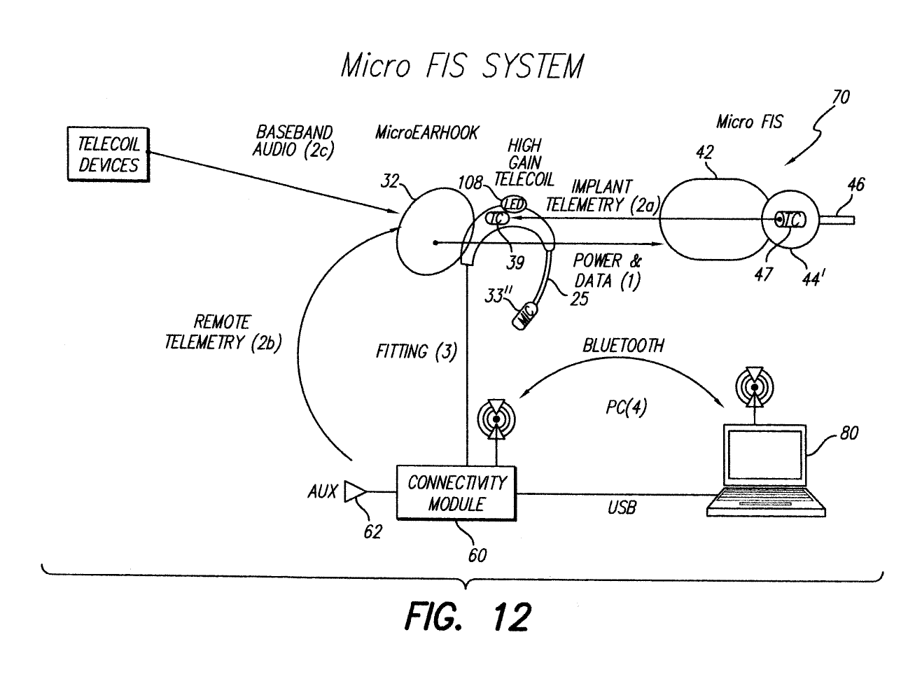

FIG. 12 schematically shows the components of an example embodiment of a Micro Fully Implantable Stimulation (FIS) system 70, and more particularly shows various communication links that can be established with such system 70. In this example embodiment, many components of the MicroFIS system 70 can be the same as those in FIG. 6, in which case the same reference numerals are used to designate such common components. The MicroEARHOOK components of the MicroFIS system 70 can be the same as those in FIG. 5, in which case the same reference numerals are used to designate such common components.

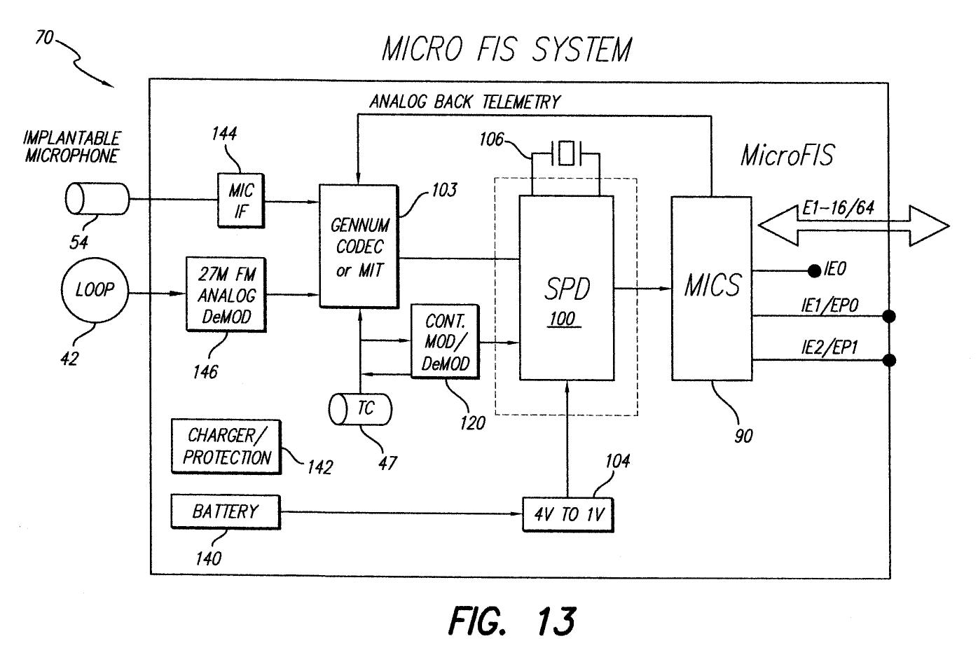

FIG. 13 is a block diagram of an example embodiment of the MicroFIS system 70. Such system 70 includes many components previously described, and such components are referred to using the same reference numerals as used previously. In this example embodiment, an implantable microphone 54 (e.g., direct, pickup, linear transformer) connects to analog front end circuitry 103 through a microphone IF circuit 144. In this example embodiment, the implantable coil 42 connects to the analog front end circuitry 103 through a 27 MHz FM Analog Demodulation circuit 146. A battery 140 provides power that is converted by a voltage converter circuit 104 (e.g., 4-to-1 volt) for use by the signal processor 100 which, in this example, is designed for 1 volt operation. A charger and protection circuit 142 is used to charge the battery 140 and to protect it from being overcharged or from being depleted to too low a charge. Other elements included within the MicroFIS system 70—e.g., the analog front end circuit 103, the 1 v signal processor 100, the continuous modulation/demodulation circuit 120, and the MICS chip 90—are as previously described. In this example embodiment, the MICS chip 90 does not need a RF receiver or transmitter.

As described above, it is thus seen that an example embodiment of the present invention provides a headpieceless and magnetless cochlear implant system (e.g., including a single external device) that offers the advantages and features as summarized below in Table 1.

```
TABLE 1
!COGS (Cost of Goods Sold)
    1.5x Reduction from Auria/HR90K
!Cosmetics
    Small BTE/No Headpiece
    Minimum Incision
!Reliability
    Reduction of Piece Parts and Connectors (e.g. No Headpiece & 4 pin
    battery/programming connector)
    Simplified Use Model (No Lock)
!Medical
    Small Implant
    No Magnet
    Minimum Incision
!Regulatory
    Legal Frequencies
!Performance
    Ultra Low Power (1+ day from a single Zinc Air Battery)
    Tiered Features
        Zinc Air or Lion (required for HiRate)
        16, 32 Contact
        Software Differentiation
    Ultra High Spatial/Frequency and Temporal Resolution
    72 dB Real-Time NRI/EABR/PAMR
!Fully Implantable
    20 Year 1 Piece System
!Accessories
    Integrated Telecoil
    Connectivity Processor
        Remote Control
        Bluetooth
        Charger
```

In various embodiments of the present invention, a cochlear stimulation apparatus includes an external device with a magnetic core which provides a low reluctance path between the external device and an implantable device of the cochlear stimulation apparatus by focusing field lines as they move from the external device toward the implantable device.

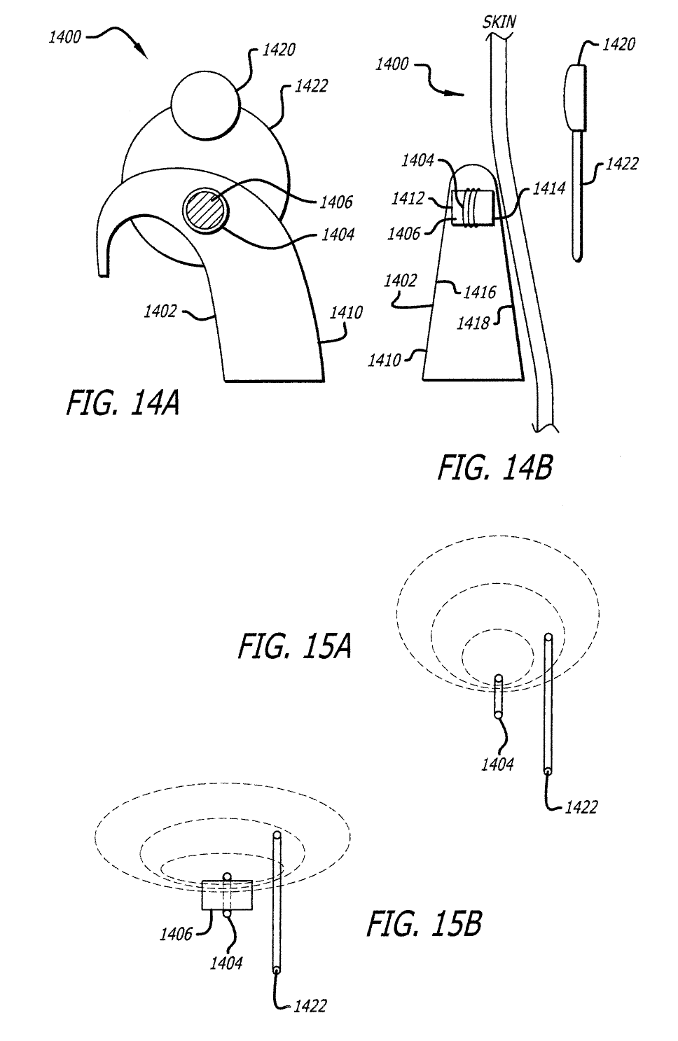

Referring to FIGS. 14A and 14B, in another example embodiment, a cochlear stimulation apparatus 1400 includes an external device 1402 with a transfer coil 1404 and a ferromagnetic core 1406. Although not shown in FIGS. 14A and 14B, the external device 1402 includes (e.g., as shown in FIG. 1) circuitry for processing sensed sound information to generate signals which are received by an implantable device 1420 of the cochlear stimulation apparatus 1400. In this example embodiment, the external device 1402 is a BTE unit; however, the external device 1402 can be provided in other configurations. In an example embodiment, the external device 1400 includes an ear hook. In an example embodiment, the external device 1400 is a single, integral unit. In this example embodiment, the implantable device 1420 includes a receiving coil 1422. Although not shown in FIGS. 14A and 14B, the implantable device 1420 includes (e.g., as shown in FIG. 1) an array of electrodes configured to be fitted within the cochlea of a user, and circuitry for receiving signals through the receiving coil and generating stimulation pulses that are directed to selected electrodes of the array.

In an example embodiment, the ferromagnetic core 1406 has a high permeability and high resistivity at the frequencies of interest, e.g., a material that is suitable at 49 MHz. By way of example, the ferromagnetic core 1406 is made of a powdered iron mixture, such as Amidon mix #67 which has a relative permeability of 40 and a resistivity of 10⁷ Ohm-cm. Such a material is suitable for applications between 10 MHz and 80 MHz. For applications at other frequencies, other magnetic materials can be selected.

In an example embodiment, the ferromagnetic core 1406 is cylindrical and has a diameter of approximately 0.25 inches. The ferromagnetic core 1406, which is used to wind the transfer coil 1404, is made small in diameter compared to the receiving coil 1422. In this example embodiment (e.g., with a distance of ≦1 cm between the ferromagnetic core 1406 and the receiving coil 1422), the required inductance is achieved because of the increased magnetic permeability. In an example embodiment, the receiving coil 1422 is sufficiently larger than the transfer coil 1404 to provide a flattened coupling coefficient response in response to an increase in distance between the coils.

It has been observed that the ferromagnetic core 1406, positioned across the external device 1400 as shown in FIG. 14B, approximately reduces the separation of the transfer coil 1404 and the receiving coil 1422 by the length of the ferromagnetic core 1406. Thus, in an example embodiment, the ends of the ferromagnetic core 1406 are positioned adjacent to (or as close as possible to) opposite inside walls of the external device 1400.

Referring to FIG. 15B (as compared to FIG. 15A, which shows the transfer coil 1404 without the ferromagnetic core 1406), the ferromagnetic core 1406 provides a low reluctance path that focuses the magnetic flux lines, thereby allowing more of the external device 1400 to be filled with conductive electronic circuits while minimizing eddy current losses. For clarity, in FIGS. 15A and 15B, the flux lines are only shown on one side of the coil axis. In this example embodiment, the ferromagnetic core 1406 forces the flux lines to be parallel in traversing through the ferromagnetic core 1406 which prevents the lines from diverging until they reach the end of the core. This effectively moves the centers of the coils closer together. The reduction in spacing increases the coupling coefficient, which allows a transfer coil 1404 with a smaller diameter to be used.

In an example embodiment, a cochlear stimulation apparatus includes an implantable device including a receiving coil, an array of electrodes configured to be fitted within the cochlea of a user, and circuitry for receiving signals through the receiving coil and generating stimulation pulses that are directed to selected electrodes of the array, and an external device in the form of a single, integral unit, the external device including circuitry for processing sensed sound information to generate the signals, the external device including a transfer coil for transferring the signals to the receiving coil and a ferromagnetic core for providing a low reluctance path between the transfer coil and the receiving coil.

In an example embodiment, a cochlear stimulation apparatus includes an implantable device including electrodes configured to be fitted within the cochlea of a user, circuitry for processing signals to generate stimulation pulses that are directed to the electrodes, and a receiving coil, and an external device including circuitry for processing sensed sound information to generate the signals, the external device including a transfer coil for communicating the signals from the external device to the implantable device, the transfer coil being inductively coupled to the receiving coil, wherein the receiving coil is sufficiently larger than the transfer coil to provide a flattened coupling coefficient response in response to an increase in distance between the coils.

In an example embodiment, the external device 1400 includes a housing 1410 that is symmetrical in shape (as shown in FIG. 14B) such that the external device 1400 can be used for either the left or right ear. In an example embodiment, the ferromagnetic core 1406 includes ends 1412, 1414 that are positioned at substantially equal distances from opposite inside walls 1416, 1418, respectively, of the housing 1410. In the example embodiment shown in FIG. 14B, the transfer coil 1404 surrounds the ferromagnetic core 1406 at a center portion of the ferromagnetic core 1404.

In an example embodiment, a cochlear stimulation apparatus includes an implanted portion, and an external portion that includes a microphone for sensing sound, a behind-the-ear (BTE) unit for enclosing electrical circuitry and a power source, the BTE unit being symmetrical in shape such that the BTE unit can be used for either the left or right ear, sound processing circuitry within the BTE unit for processing signals generated by the microphone in response to sound sensed through the microphone or otherwise applied to the sound processing circuitry as an input signal, signal processing circuitry within the BTE unit for processing the input signal and generating stimulation, control and power signals for transferring to the implanted portion, and an external coil within the BTE unit for coupling the stimulation, control and power signals to the implanted portion.

In an example embodiment, the implanted portion includes an implanted coil inductively coupled with the external coil, electronic circuitry for receiving through the implanted coil the stimulation, control and power signals, an electrode array having a multiplicity of electrode contacts adapted to be placed within the cochlea of a user, and a pulse generator for generating stimulation pulses that are directed to selected electrode contacts within the electrode array as controlled by the control signals.

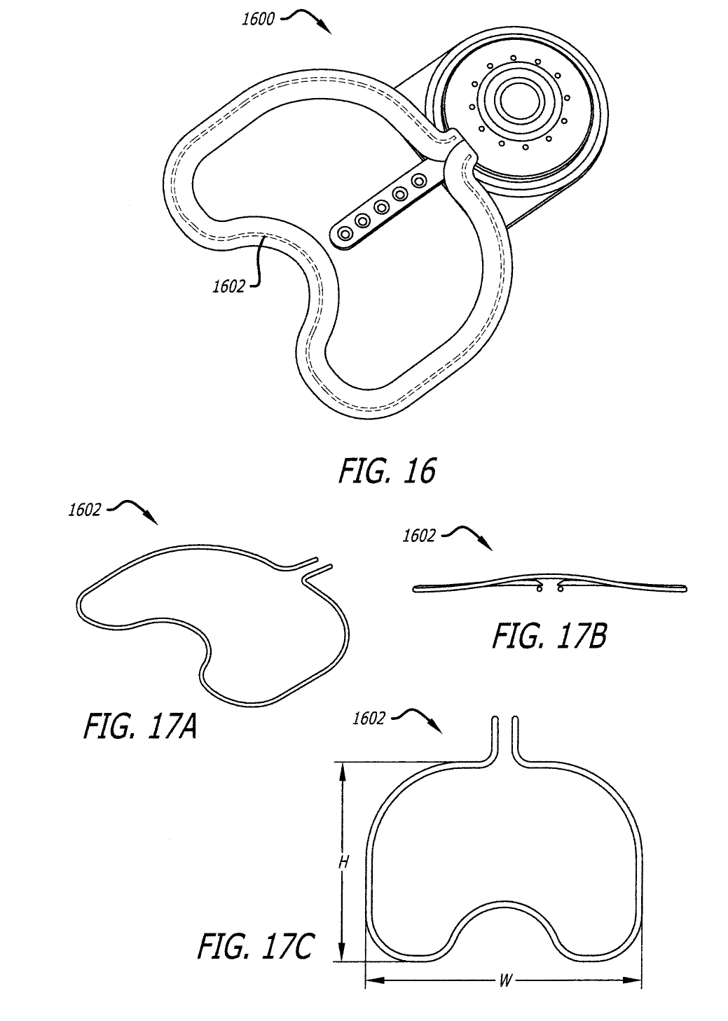

Referring to FIG. 16, in an example embodiment, an implantable device 1600 includes a D-shaped receiving coil 1602 encased in a protective barrier 1604. In this example embodiment, the implanted coil can be described as having two symmetrical lobes. The symmetrical shape allows the implantable device 1600 to be flipped for left or right ear use. In an example embodiment, and referring to FIGS. 17A-17C, the D-shaped receiving coil 1602 has a width (W) of 35 mm and a height (H) of 25 mm. It should be appreciated that the receiving coil 1602 can have other dimensions as well as other shapes. Moreover, the receiving coil 1602 does not have to be symmetrical in shape.

While the invention herein disclosed has been described by means of specific embodiments and applications thereof, numerous modifications and variations could be made thereto by those skilled in the art without departing from the scope of the invention set forth in the claims.

What is claimed is:

1. A cochlear stimulation apparatus comprising:
an implantable device including a receiving coil, an array of electrodes configured to be fitted within the cochlea of a user, and circuitry for receiving signals through the receiving coil and generating stimulation pulses that are directed to selected electrodes of the array; and
an external device in the form of a single, integral unit, the external device including circuitry for processing sensed sound information to generate the signals, the external device including a transfer coil for transferring the signals to the receiving coil and a ferromagnetic core for providing a low reluctance path between the transfer coil and the receiving coil.

2. The cochlear stimulation apparatus of claim 1, wherein the external device includes a housing that is symmetrical in shape such that the external device can be used for either the left or right ear.

3. The cochlear stimulation apparatus of claim 2, wherein the ferromagnetic core includes ends that are positioned at substantially equal distances from opposite inside walls of the housing.

4. The cochlear stimulation apparatus of claim 1, wherein the transfer coil surrounds the ferromagnetic core at a center portion of the ferromagnetic core.

5. The cochlear stimulation apparatus of claim 1, wherein the ferromagnetic core has a length of approximately 1 cm or less.

6. The cochlear stimulation apparatus of claim 1, wherein the receiving coil includes two symmetrical lobes.

7. The cochlear stimulation apparatus of claim 1, wherein the receiving coil is larger than the transfer coil.

8. The cochlear stimulation apparatus of claim 7, wherein the size disparity between the coils is sufficiently large to provide a flattened coupling coefficient response in response to an increase in distance between the coils.

9. The cochlear stimulation apparatus of claim 1, wherein the external device includes a behind-the-ear (BTE) unit.

10. The cochlear stimulation apparatus of claim 9, wherein the transfer coil is contained within the BTE unit.

11. A cochlear stimulation apparatus comprising:
an implanted portion; and
an external portion that includes
a microphone for sensing sound,
a behind-the-ear (BTE) unit for enclosing electrical circuitry and a power source, the BTE unit being symmetrical in shape such that the BTE unit can be used for either the left or right ear,
sound processing circuitry within the BTE unit for processing signals generated by the microphone in response to sound sensed through the microphone or otherwise applied to the sound processing circuitry as an input signal,
signal processing circuitry within the BTE unit for processing the input signal and generating stimulation, control and power signals for transferring to the implanted portion, and
an external coil within the BTE unit for coupling the stimulation, control and power signals to the implanted portion.

12. The cochlear stimulation apparatus of claim 11, wherein the BTE unit includes an earhook.

13. The cochlear stimulation apparatus of claim 11, wherein the external portion includes a ferromagnetic core for providing a low reluctance path between the external coil and the implanted portion.

14. The cochlear stimulation apparatus of claim 13, wherein the ferromagnetic core includes ends that are positioned at substantially equal distances from opposite inside walls of the BTE unit.

15. The cochlear stimulation apparatus of claim 13, wherein the external coil surrounds the ferromagnetic core at a center portion of the ferromagnetic core.

16. The cochlear stimulation apparatus of claim 13, wherein the ferromagnetic core has a length of approximately 1 cm or less.

17. The cochlear stimulation apparatus of claim 11, wherein the implanted portion includes:
an implanted coil inductively coupled with the external coil;
electronic circuitry for receiving through the implanted coil the stimulation, control and power signals;
an electrode array having a multiplicity of electrode contacts adapted to be placed within the cochlea of a user; and
a pulse generator for generating stimulation pulses that are directed to selected electrode contacts within the electrode array as controlled by the control signals.

18. The cochlear stimulation apparatus of claim 17, wherein the implanted coil includes two symmetrical lobes.

19. The cochlear stimulation apparatus of claim 17, wherein the implanted coil is larger than the external coil.

20. The cochlear stimulation apparatus of claim 19, wherein the size disparity between the coils is sufficiently large to provide a flattened coupling coefficient response in response to an increase in distance between the coils.

21. A cochlear stimulation apparatus comprising:
an implantable device including electrodes configured to be fitted within the cochlea of a user, circuitry for processing signals to generate stimulation pulses that are directed to the electrodes, and a receiving coil; and
an external device including circuitry for processing sensed sound information to generate the signals, the external device including a transfer coil for communicating the signals from the external device to the implantable device, the transfer coil being inductively coupled to the receiving coil;
wherein the receiving coil is sufficiently larger than the transfer coil to provide a flattened coupling coefficient response in response to an increase in distance between the coils.

22. The cochlear stimulation apparatus of claim 21, wherein the external device includes a ferromagnetic core for providing a low reluctance path between the transfer coil and the receiving coil.

23. The cochlear stimulation apparatus of claim 22, wherein the transfer coil surrounds the ferromagnetic core at a center portion of the ferromagnetic core.

24. The cochlear stimulation apparatus of claim 22, wherein the ferromagnetic core has a length of approximately 1 cm or less.

25. The cochlear stimulation apparatus of claim 22, wherein the ferromagnetic core includes ends that are positioned at substantially equal distances from opposite inside walls of the external device.

26. The cochlear stimulation apparatus of claim 21, wherein the receiving coil includes two symmetrical lobes.

27. The cochlear stimulation apparatus of claim 21, wherein the external device includes a housing that is symmetrical in shape such that the external device can be used for either the left or right ear.

28. The cochlear stimulation apparatus of claim 21, wherein the external device includes a behind-the-ear (BTE) unit.

29. The cochlear stimulation apparatus of claim 21, wherein the external device includes an ear hook.

30. The cochlear stimulation apparatus of claim 21, wherein the external device is a single, integral unit.
